(19) United States
(12) Patent Application Publication
(10) Pub. No.: US 2012/0108507 A1
(43) Pub. Date: May 3, 2012

(54) Methods for In Vivo Cell Reprogramming

(75) Inventors: Yong Zhu, Anaheim, CA (US); Shili Wu, Edmond, OK (US); Jun Bao, Rowland Heights, CA (US)

(73) Assignee: VIVOSCRIPT, INC., Costa Mesa, CA (US)

(21) Appl. No.: 13/288,040

(22) Filed: Nov. 3, 2011

Related U.S. Application Data
(60) Provisional application No. 61/409,946, filed on Nov. 3, 2010.

(51) Int. Cl. A61K 38/02; A61K 31/7088; A61P 3/10; A61P 9/00; G01N 33/53; A61P 25/16; A61P 25/28; C12Q 1/02; C12Q 1/68; C12N 5/071; A61P 19/00

(52) U.S. Cl. 514/6.9; 435/377; 514/44 R; 514/17.8; 514/17.7; 514/1.1; 435/29; 435/6.12; 435/7.1

(57) ABSTRACT

The present disclosure provides a new technology platform that converts one type of cells (substrate cells) to another type of cells (product cells).

### BACKGROUND

Embryonic stem cells (ESCs) are capable of differentiating into every type of cells in human bodies. Differentiation from ESCs to progenitor cells and eventually to somatic cells was originally believed to be one-way street. There was no turning back. The majority of somatic cells are terminally differentiated and were believed to lack the capability of changing to other types of somatic cells. In 2006, this old paradigm was toppled when Dr. Yamanaka and his team made the sensational discovery of induced Pluripotent Stem Cell (iPSC): Somatic cells were reprogrammed to pluripotent stem cells via ectopic expression of four transcription factors, i.e. Oct4, Klf4, Sox2 and c-Myc via viral transduction (1). In 2008, Dr. Melton's group demonstrated that pancreatic exocrine cells can be transdifferentiated to β cells in vivo via virus transduction of genes of several pancreatic and β cell transcription factors, including Ngn3 (Neurog3), Pdx1, and MafA (2). As these and other studies indicate, cell fate is not fixed. It is possible that somatic cells can be reversed back to pluripotent stem cells, or be directly converted to another type of somatic cells, with forced expression of several transcription factors.

It was reported that mouse fibroblast cells were able to be converted to iPSCs without the use of any form of exogenous genetic material (3). Four transcription factor proteins—oct4, sox2, klf4, and c-myc—which are capable of penetrating into and reprogramming cells were engineered and produced. By applying recombinant proteins, the risk of modifying the target cell genome eliminated, making it possible to use iPSC and its derivative cells for therapeutic purpose.

Although great advances have been made in biomedical research, scientists and clinicians are still looking for effective treatments for some of the most devastating diseases, such as diabetes, cardiovascular diseases, spinal cord injury, Parkinson's disease, and Alzheimer's disease. Some traditionally incurable diseases are listed in Table 1. The majority of these diseases are caused by or symptomized by damage or deficiency or depletion of certain type of cells.

TABLE 1
Some incurable diseases and their associated deficient cell types.

| Disease | Deficient cell type |
|---|---|
| Diabetes | β cells |
| Myocardial Infarction | Cardiomyocytes |
| Stroke | Neurons |
| Traumatic brain injury | Neurons |
| Parkingson's disease | Dopaminergic neurons |
| Alzheimer's disease | Neurons |

Potential treatments for these diseases include drug-based therapies and cell-based therapies. Both have their limitations. Drug-based therapies usually treat symptoms only and make patients chronically dependent on them. Cell-based therapies are hampered by the scarcity of the source, immune rejection, high costs in manufacture and distribution, and high risks in development and commercialization (Table 2).

TABLE 2
The limitations of drug-based therapies and cell-based therapies.

| | Conventional Drug- | |
|---|---|---|
| | based Therapies | Cell-based Therapies |
| Modalities | Small molecules, proteins, | Cells (including stem cells |
| | RNAs . . . | or their derivatives), tissues, |
| | | or organs |
| Limitations | Some diseases are untreatable | Scarcity of donors |
| | Treat or manage symptoms only | Immune rejections |
| | Chronic dependence on drugs | Unsustainable business |
| | Expensive | models |
| | | Manufacture and |
| | | distribution costs |
| | | Regulatory risks |
| | | Development risks |
| | | Avoided by investors |

Thus there are needs to develop news measures to treat diseases associated with cell damage, depletion or deficiency.

### SUMMARY

In one aspect, the present disclosure provides a method of converting a substrate cell to a product cell comprising: selecting at least one reprogramming factor capable of converting the substrate cell to the product cell; and applying the reprogramming factor to the substrate cell.

In certain embodiments, the reprogramming factor is selected based on gene regulatory network and selection criteria.

In certain embodiments, the reprogramming factor is selected from the group consisting of protein, small molecule, and small RNA.

In certain embodiments, the substrate cell and the product cell pair is selected from the group consisting of a liver/pancreatic exocrine cell and beta cell pair; a T cell and a regulatory T cell pair; a white fate cell and a brown fat cell pair; an astrocyte and a neuron pair; and a cardiac fibroblast and a cardiomyocyte pair.

In another aspect, the present disclosure provides a method of treating a disease associated with deficiency, damage or depletion of a cell type comprising: applying at least one reprogramming factors to a substrate cell; and converting the substrate cell into a product cell, wherein the product cell is the cell type deficient, damaged or depleted in the disease.

In certain embodiments, the substrate cell is abundant in and in proximity to the site of the disease and capable of being converted to the product cell.

In certain embodiments, the disease is selected from the group consisting of diabetes, cardiovascular diseases, spinal cord injury, Parkinson's disease, and Alzheimer's disease.

In certain embodiments, the reprogramming factor is selected from the group consisting of protein, small molecule, and small RNA.

In certain embodiments, the reprogramming factor is selected based on gene regulatory network and selection criteria.

In certain embodiments, the present disclosure provides a method for identifying reprogramming factors which are capable of converting one type of cells to another type, comprising: identifying kernel modules or proteins in Gene Regulatory Network wherein the proteins are homologous among difference species and conservative in both amino acid sequences and cis-regulatory sequences to which the kernel protein bind to or are regulated.

In certain embodiments, the present disclosure provides a computer readable storage medium having a computer program product encoded thereon, wherein said computer program product when executed by a computer instructs the computer to execute the method for identifying reprogramming factors which are capable of converting one type of cells to another type.

### BRIEF DESCRIPTION OF THE FIGURES

FIG. 1 shows the basic model for in vivo reprogramming.

FIG. 2 shows mechanisms of cell reprogramming. FIG. 2 A shows the linage tree view; FIG. 2B shows the stochastic model view.

FIG. 3 shows the Gene Regulatory Network of beta cells.

FIG. 4 shows the Gene Regulatory Network of Heart.

FIG. 5 shows immunofluorescent analysis of liver tissues of mice treated with control protein or with Vivo-6, -7, and -8 proteins.

FIG. 6 shows immunofluorescent analysis of pancreatic tissues of mice treated with control protein or with Vivo-6, -7, and -8 proteins.

FIG. 7 shows the projected role of in vivo cell reprogramming therapies. The size of the area enclosed by each circle indicates how big the market for that type of treatment is. The dotted circle is the projected market for in vivo cell reprogramming therapies.

FIG. 8 shows a schematic drawing for the process of using qPCR to screen the reprogramming factor combinations.

### DETAILED DESCRIPTION OF THE INVENTION

The present disclosure provides a new technology platform, called In Vivo Cell Reprogramming, or IVR. Using a combination of reprogramming factors (e.g., protein drugs), one type of cells (substrate cells) can be converted to another type of cells (product cells). Product cells are usually the damaged or depleted or deficient cells associated with certain diseases, and substrate cells are one type of cells that are abundant in the proximity of or in the affected organs. For example, in one IVR application, liver cells or acinar cells (substrate cells) can be converted to 13 cells (product cells) to treat diabetes.

IVR combines the advantages of drug-based and cell-based therapies while avoiding the shortfalls of both. Since the reprogramming factors of IVR therapeutics are normally proteins, their risk profiles and cost structures of development and commercialization are similar to those of drug-based therapies. However, by using a cell-based mechanism, IVR therapeutics can achieve therapeutic benefits that cannot be achieved by conventional drugs. In addition, the newly created product cells are sustainable. Therefore a short course of treatment can provide long-term clinical benefits. Moreover, contrary to most situations in cell or tissue transplantation, new product cells will not cause immune rejection. WR can be applied to several diseases which are caused by or symptomized by damage or deficiency or depletion of certain type of cells. The examples of the disease are listed in Table I which includes diabetes, autoimmune diseases, and stroke.

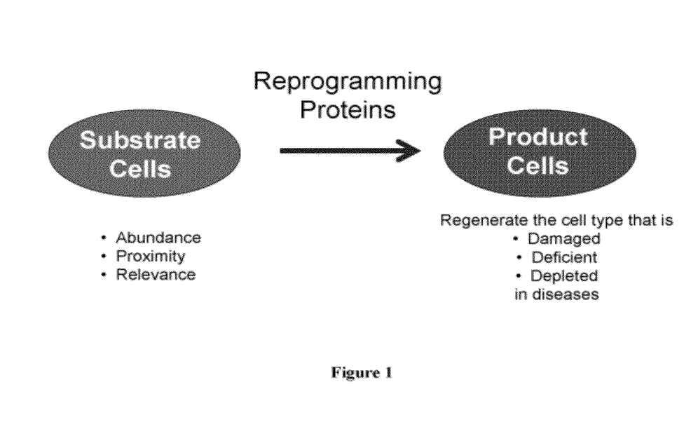

The IVR disclosed in the present disclosure, also named as 4R—cell regeneration, replacement, repair, and rejuvenation, provides solutions to regenerative medicine. Using reprogramming factors (e.g., a combination of protein drugs and/or small molecule drugs), substrate cells can be converted to product cells as shown in FIG. 1.

As disclosed, product cells are usually the damaged or depleted cells associated with certain diseases, and substrate cells are one type of cells that are abundant and in the proximity of or in the affected organs. Another criterion for selecting substrate cells is relevance—its lineage should be very close to product cells' lineage in terms of development and differentiation. These three criteria—abundance, proximity, and relevance—not only make the reprogramming easier, but also ensure that the newly created product cells function similarly as the original cells in the same microenvironment and in response to the same physiological signals.

For example, in one IVR application, liver cells or acinar cells (substrate cells) can be converted to 13 cells (product cells) to treat diabetes. More examples of the pairs of substrate and product cells are given in Table 3.

TABLE 3
Selection of substrate and product cells in targeting some diseases.

| Disease | Substrate Cell | Product Cell |
|---|---|---|
| Diabetes | liver cells | β cell |
| | pancreatic exocrine cells | |
| Autoimmune diseases | T cells | Regulatory T cells |
| Obesity | White fat cells | Brown fat cells |
| Stroke | Astrocytes | Neurons |
| Myocardial infarction | Cardiac fibroblasts | Cardiomyocytes |

Reprogramming factors can be proteins, small molecules, and small RNAs (including siRNAs and miRNAs). The proteins include transcription factors and chromosome remodeling proteins. In certain embodiment, reprogramming factors are proteins. The reprogramming proteins should be able to get into the nuclei to be functional, but normally in their native forms they cannot enter cells. Thus, in certain embodiments, the engineered proteins as reprogramming factors are recombinant proteins that comprising protein transduction domains (PTDs) which are fused to functional unit of the proteins. As a result, the engineered proteins are transducible, i.e., capable of penetrating cells and relocating into nuclei when placed in cell culture medium or around the affected organs.

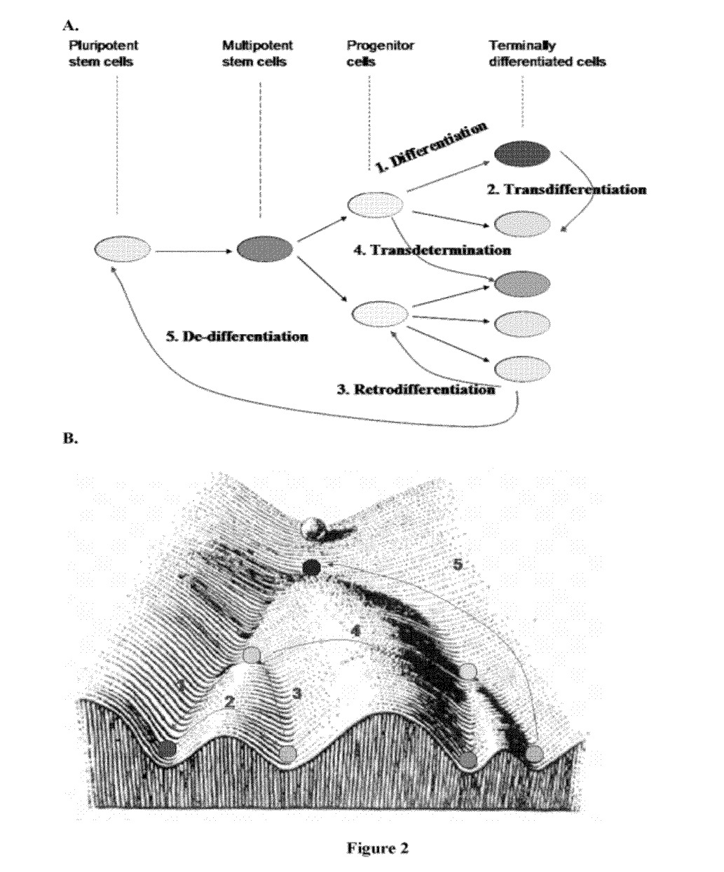

The conversion from the substrate cells to the product cells can be through any of the five mechanisms: 1) Differentiation, 2) Transdifferentiation, 3) Retrodifferentiation, 4) Transdetermination, and 5) Dedifferentiation. These mechanisms are illustrated in FIG. 2.

FIG. 2A is a view of cell differentiation using a simplified cell lineage tree. FIG. 2B is adapted from the differentiation model proposed by Conrad Waddington 50 years ago. The ball sitting atop a slope and poised to roll down into different branching ravines represents the totipotent zygote ready to differentiate to pluripotent and multipotent stem cells and somatic cells and to take different fates.

The present disclosure also relates to the selection of a reprogramming factor or a combination of reprogramming factors that convert a substrate cells to a product cell or the identification of reprogramming factors in a given pair of the substrate and product cells.

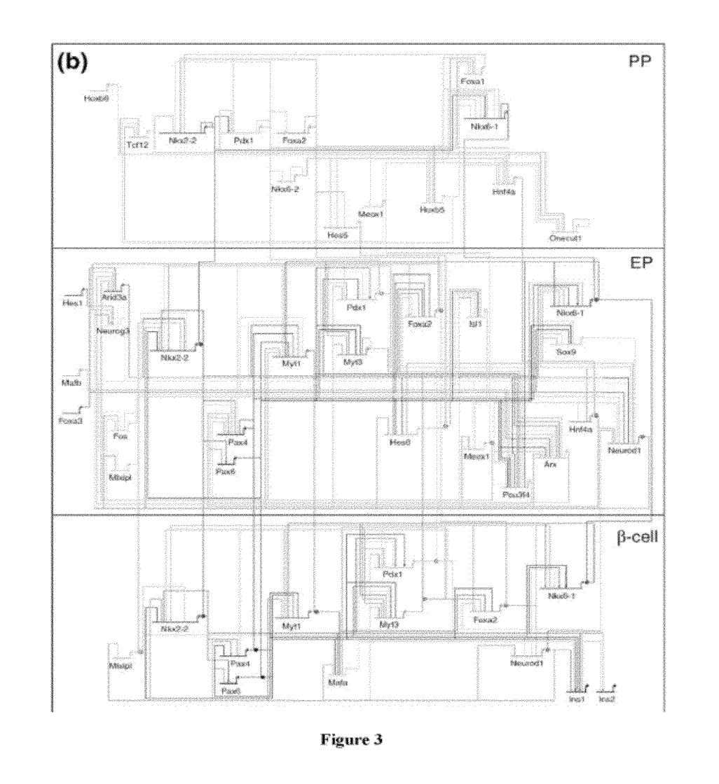

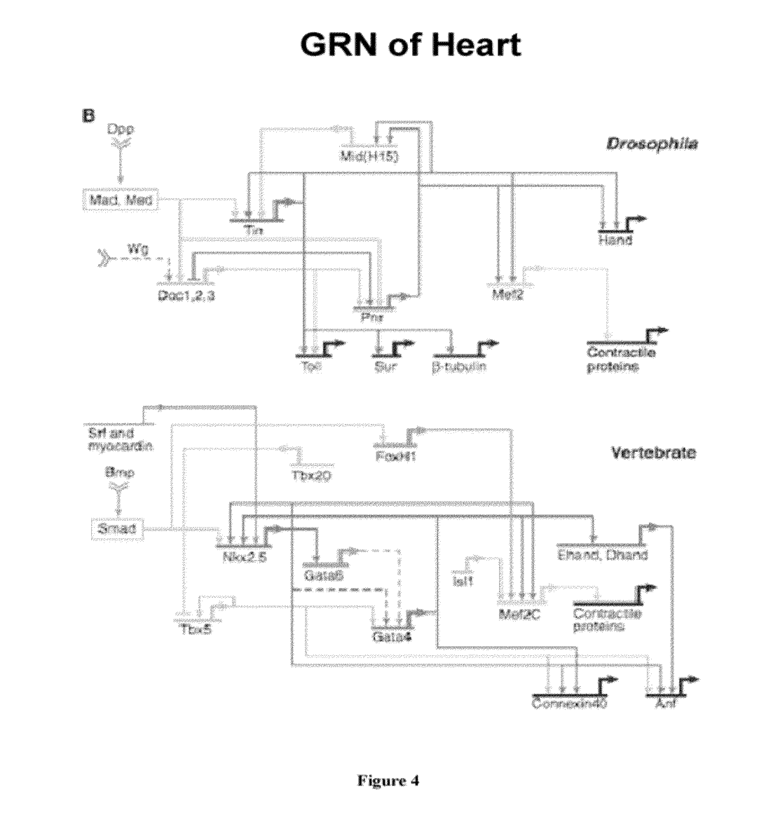

The development process from a single cell to a complicated individual is hard-wired in the species' genome—the pathways to different cell fates are encoded by Gene Regulatory Network (GRN) (4). GRN is an extensive network that consists of hundreds of thousands of genes and their products (nodes) and the regulatory relations among them (edges) (See FIG. 3, GRN of beta cells; and FIG. 4, GRN of heart cells). Although all the cells in human body share one GRN, the state of GRN differs significantly from one type of cells to the next. Some parts of the GRN are active or lightened up in (3 cells, for example, but are inactive or go dark in liver cells, and vice versa. In fact, what differentiates β cells from liver cells is its internal state of the GRN. The differences in GRN states are due to differences in cell environment, the legacy components from parent cells, and the history of cell lineages. So the reprogramming process is to perturb one state of the GRN strongly enough to jolt it into another state, or to turn on parts of the GRN while simultaneously shut down the other parts. Transcription factors are the convenient choices because they are the most important components of the GRN.

Comparing the GRN states of two different types of cells or two different species uncovers hundreds of transcription factors that differ significantly in expression. But there is a shortcut to cut through the maze. Among all of these nodes exists an active kernel that is highly conserved in the same type of cells among different species. Some nodes of such conservative kernel in the product cells may be inactive in the substrate cells. The protein products of such nodes are instrumental in the conversion process, as kernels are very conservative among species that lead to a cascade of subsequent protein expressions or cell fates. In addition, the amino acids sequence of a kernel protein among various species share conservative sequences and homologies. The cis-regulatory nucleotide sequences or regions of the kernel protein or the regions that the kernel protein bind to are also substantially homologous or conservative among various species. For example, in FIG. 4, “HAND” in Drosophillia is very similar to “EHAND” in vertebrate.

Another key feature of the active kernel module for a specific cell is that the module enters a “lock on” state once the cells' fate is decided. The “lock on” state is maintained through several positive feedback loops or self-sustainable mechanism. Therefore after introducing several external key transcription factor proteins and initiating a new GRN state, the external reprogramming factors may no longer be required once the a new GRN is initiated. Even when the reprogramming proteins are withdrawn from the environment, the cells will maintain the new fate because the new GRN state is maintained internally and becomes very stable, i.e. “locked on”.

It is also contemplated that the kernel module is capable of amplifying the signal and leading to a cascade of amplification of reaction. In certain embodiment, the kernel module tends to be at the upstream of the GRN or a cell fate and substantially less expressed in substrate cells in comparison to the product cells.

In general, the selection criteria for reprogramming factors include, but are not limited to, identification kernel modules or proteins in GRN wherein the proteins are homologous among difference species and conservative in both amino acid sequences and cis-regulatory sequences to which the kernel protein bind to or are regulated. The external reprogramming factors are preferably capable of amplifying the cascade of regulatory events. Once the external factors are administered, the factors are preferably capable of triggering self-sustainable, positive feed back loop such that additional factors may not be required to maintain the conversion. In addition, the factors are substantially lacking or less expressed in the substrate cells in comparison to the product cells.

In certain embodiments, the present disclosure provides a computer program product comprising one or more instructions recorded on a machine-readable recording medium for identifying reprogramming factors which are capable of converting one type of cells to another type, wherein the one or more instructions are for identifying kernel modules or proteins in Gene Regulatory Network wherein the proteins are homologous among difference species and conservative in both amino acid sequences and cis-regulatory sequences to which the kernel protein bind to or are regulated.

In certain embodiments, the present disclosure provides a computer readable storage medium having a computer program product encoded thereon, wherein said computer program product, when executed by a computer, instructs the computer to execute the method for identifying reprogramming factors which are capable of converting one type of cells to another type.

In certain embodiments, the computer readable storage medium may be any of a variety of memory storage devices. Examples of memory storage medium include any commonly available random access memory (RAM), magnetic medium such as a resident hard disk or tape, an optical medium such as a read and write compact disc, or other memory storage device.

### EXAMPLES

### Example 1

To illustrate, the IVR technology is used to treat diabetes. The strategy is to reprogram liver and pancreatic exocrine cells to insulin-producing beta cells using a combination of proteins: protein Vivo-6 (Pdx1, SEQ ID NO: 1), -7 (Ngn3, SEQ ID NO: 2), and -8 (MafA, SEQ ID NO: 3). Below are some preliminary data.

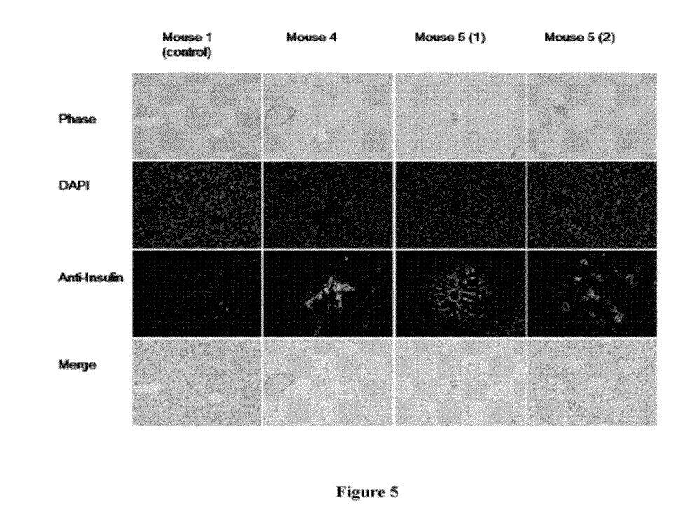

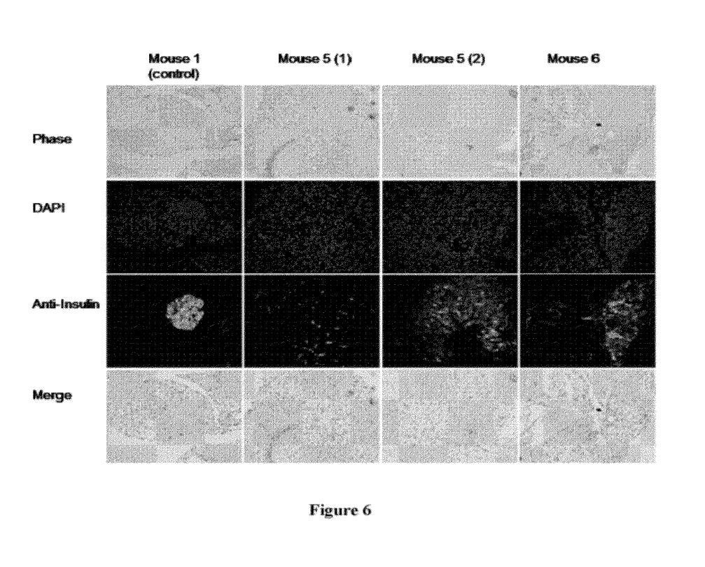

Six CD-1 mice were divided into two groups: the treatment group and the control group. Transducible protein Vivo-6 (1 mg/kg), Vivo-7 (1 mg/kg), and Vivo-8 (1 mg/kg) were injected into each mouse by intraperitoneal (IP) in treatment group (Mouse-4, Mouse-5 and Mouse-6) and BSA (1 mg/kg) was injected into each mouse in the control group (Mouse-1, Mouse-2 and Mouse-3). Injections were repeated every day for 7 days. Mice of both treatment and control group were sacrificed on day 10, 3 days after completion of injections. The mouse liver and pancreas were washed with 1×PBS and fixed by 4% paraformaldehyde for overnight. Then the liver and pancreatic tissues were processed by standard Paraffin Embedding protocol. The Tissue sections, 5-micro in thickness, were prepared routinely with histology microtomes and mounted on standard histology glass slides. The wax in tissues was dissolved by xylene during processing of tissue sections. Tissue sectioning and histologic and immunohistochemical staining were performed using routine methods. For indirect fluorescent-antibody (IFA) assay, the slides were blocked with 0.05% Tween 20 (TBST) and 3% BSA for 1 hour at RT and were incubated with mouse anti-insulin antibody (Invitrogen) at 4° C. overnight. The slides were washed three times with PBS for 15 minutes at RT and incubated with fluorescein isothiocyanate (FITC) conjugated swine anti-mouse antibody (KPL) for 2 hours at RT. Same concentration of Mouse IgG was used as isotype control. Anti DAPI antibody was added to slides as a nuclear marker (FIGS. 5-6).

The results showed that the treatment group had more insulin-producing cells in livers compared with the control group (FIG. 5). There are some insulin positive cells in the liver of control animals (mouse 1, FIG. 5). This observation is not surprising since the same kind of leakiness has been reported in the literature before. In the livers of protein-treated mice, clusters of insulin-positive cells emerged, many of which are concentrated in the periportal areas.

In the pancreas of mice treated with the three transducible proteins, clusters of insulin-producing cells were observed besides the normal insulin-intensive β islets. Different from the native β islets, these clusters of insulin-producing cells are more mosaic and diffusive (FIG. 6).

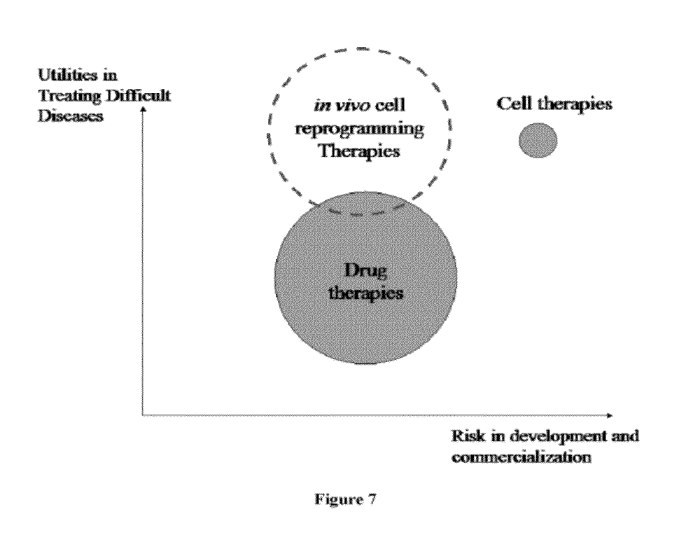

In general, IVR potentially offers the advantages of drug-based and cell-based, therapies while avoiding the shortfalls of both. Since the modality of IVR therapeutics are proteins, their risk profiles and cost structures of development and commercialization are similar to those of drug-based therapies (FIG. 7). However, by using a cell-based mechanism, IVR therapeutics can achieve therapeutic benefits that cannot be achieved by conventional drugs. In addition, the newly created product cells are sustainable. Therefore a short course of treatment can provide long-term clinical benefits. Moreover, contrary to most situations in cell or tissue transplantation, new product cells will not cause immune rejection.

### Example 2

Here we provide an example demonstrating how to apply the IVR principles to identifying the reprogramming factors in treating diabetes.

To regenerate the missing cells in diabetic patients, we select pancreatic exocrine cells as substrate cells and cells as product cells. By searching and compiling the literature, we draw maps of GRNs for both pancreatic exocrine cells and cells. An example of the GRN state of cells is illustrated in the lower panel of FIG. 3. By comparing and studying the GRN maps of pancreatic exocrine cells and cells, with an emphasis on the GRN state of cells, we come up with a list of candidate reprogramming factors. Based on the topology and the logic of the GRN state of cells, the Pdx1, Ngn3, Nkx-2.2, Nkx-6.1, and MafA are selected as key factors that maintain specific gene battery and phenotype for cells. They are good candidates as reprogramming factors.

Design and synthesize the genes for the potential reprogramming factors. Their genes are fused in frame with the ones of protein transduction domains or supercharged GFPs, so that the expressed recombinant proteins are cell transducible, i.e., they can penetrate into cells once placed in cell medium or inside animal bodies. The proteins are expressed and purified from E. coli transformed with those synthesized genes.

To further confirm and narrow down the optimal reprogramming factors, we use an in vitro model to screen different combinations. A rat pancreatic acinar cell line—AR42J—is selected. The cells are cultured and treated with different combination of transducible reprogramming proteins including Pdx1, Ngn3, Nkx-2.2, Nkx-6.1, and MafA at various time points at different doses. Small molecule chemicals and/or cytokines are added to facilitate the reprogramming progress. Then the cells are lysed, their RNA extracted, and cDNA reverse-transcribed.

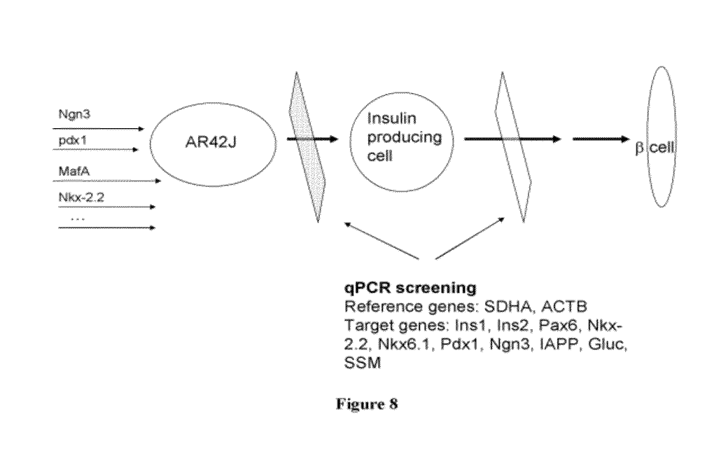

To select the best reprogramming condition, qPCR is used to quantitatively measure the changes in the mRNA levels of specific genes that are expressed in cells, such as Ins1, Ins2, Pax6, Nkx-2.2, Nkx6.1, Pdx1, Ngn3, IAPP, Gluc, and SSM (FIG. 8, see Table 4 for more description for the genes). For relative quantification purpose, several housekeeping genes such as SDHA and ACTB are also measured (see Table 4 for more description for the genes). Genes that are specifically expressed in the other types of endocrine cells such as glucagon and somatostatin are also measured.

TABLE 4
List of target and reference genes as biomarkers used to monitor the
reprogramming process towards beta cells.

| Gene | | |
|---|---|---|
| name | Description of the Gene | Gene Accession Number |
| Ins1 | RAT Insulin-1 Precursor | UniProtKB/Swiss-Prot P01322 |
| Ins2 | RAT Insulin-2 Precursor | UniProtKB/Swiss-Prot P01323 |
| Pax6 | RAT Paired box protein Pax-6 | UniProtKB/Swiss-Prot; |
| | (Oculorhombin) | Acc: P63016 |
| ACTB | RAT Actin, cytoplasmic 1 | UniProtKB/Swiss-Prot; |
| | (Beta-actin), contains Actin, | Acc: P60711 |
| | cytoplasmic 1, N-terminally | |
| | processed | |
| Nkx-2.2 | Rat NK2 homeobox 2 | NCBI Reference Sequence: |
| | | NM_001191904.1 |
| Nkx-6.1 | Rat NK6 homeobox 1 | NCBI Reference Sequence: |
| | | NM_031737.1 |
| IAPP | RAT Islet amyloid polypeptide | UniProtKB/Swiss-Prot; |
| | Precursor (Amylin), | Acc: P12969 |
| | Diabetesassociated peptide)(DAP) | |
| Gluc | RAT Glucagon Precursor | UniProtKB/Swiss-Prot; |
| | | Acc: P06883 |
| SMS | RAT Somatostatin Precursor | UniProtKB/Swiss-Prot; |
| | | Acc: P60042 |
| Pdx1 | RAT pancreatic and duodenal | NCBI reference: NM_022852.3 |
| | homeobox 1 | |
| Ngn3 | RAT neurogenin 3 | NCBI reference: NM_021700.1 |
| SDHA | RAT succinate dehydrogenase | NCBI reference: NM_130428.1 |
| | complex, subunit A, | |
| | flavoprotein (Fp) | |

By monitoring the trend of changes in mRNA expression of aforementioned genes, we can select the best combinations and working conditions. They are further tested in animal models, as shown in FIGS. 5 and 6, and eventually in diseased animal models to examine their efficacy in treating diabetes.

### REFERENCES

All references are incorporated herein in their entirety by reference.
1. Takahashi, K. & Yamanaka, S. Induction of Pluripotent Stem Cells from Mouse Embryonic and Adult Fibroblast Cultures by Defined Factors. 126, 663-676 (2006).
2. Zhou, Q., Brown, J., Kanarek, A., Rajagopal, J. & Melton, D. A. In vivo reprogramming of adult pancreatic exocrine cells to β-cells. Nature 455, 627-632 (2008).
3. Hongyan Zhou, Shili Wu, Jin Young Joo, Saiyong Zhu, Dong Wook Han, Tongxiang Lin, Sunia Trauger, Geoffery Bien, Susan Yao, Yong Zhu, Gary Siuzdak, Hans R. Schöler, Lingxun Duan, Sheng Ding, Generation of Induced Pluripotent Stem Cells Using Recombinant Proteins. Cell Stem Cell 4, 581 (2009).
4. Eric H. Davidson, The Regulatory Genome: Gene Regulatory Networks in Development and Evolution. Academic Press (2006).

What is claimed is:

1. A method of converting a substrate cell to a product cell comprising:
selecting at least one reprogramming factor capable of converting the substrate cell to the product cell; and
applying the reprogramming factor to the substrate cell.

2. The method of claim 1 wherein the reprogramming factor is selected based on gene regulatory network and selection criteria.

3. The method of claim 1 wherein the reprogramming factor is selected from the group consisting of protein, small molecule, and small RNA.

4. The method of claim 1 wherein the substrate cell and the product cell pair is selected from the group consisting of a liver/pancreatic exocrine cell and beta cell pair; a T cell and a regulatory T cell pair; a white fat cell and a brown fat cell pair; an astrocyte and a neuron pair; and a cardiac fibroblast and a cardiomyocyte pair.

5. A method of treating a disease associated with deficiency, damage or depletion of a cell type comprising:
applying at least one reprogramming factors to a substrate cell; and
converting the substrate cell into a product cell, wherein the product cell is the cell type deficient, damaged or depleted in the disease.

6. The method of claim 5 wherein the substrate cell is abundant in and in proximity to the site of the disease and capable of being converted to the product cell.

7. The method of claim 5 wherein the disease is selected from the group consisting of diabetes, cardiovascular diseases, spinal cord injury, Parkinson's disease, and Alzheimer's disease.

8. The method of claim 5 wherein the reprogramming factor is selected from the group consisting of protein, small molecule, and small RNA.

9. The method of claim 5 wherein the reprogramming factor is selected based on gene regulatory network and selection criteria.

10. A method for identifying reprogramming factors which are capable of converting one type of cells to another type, comprising:
identifying kernel modules or proteins in Gene Regulatory Network wherein the proteins are homologous among difference species and conservative in both amino acid sequences and cis-regulatory sequences to which the kernel protein bind to or are regulated.

11. A computer readable storage medium having a computer program product encoded thereon, wherein said computer program product when executed by a computer instructs the computer to execute the method of claim 10.


SEQUENCE LISTING (3 sequences)

SEQ ID NO: 1 - protein, 323 aa, Artificial Sequence
  Transducible material
MGSSHHHHHHSSGLVPRGSHMNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPRESGGGGSPGRRRRRRRRRRR

SEQ ID NO: 2 - protein, 254 aa, Artificial Sequence [sequence omitted]

SEQ ID NO: 3 - protein, 392 aa, Artificial Sequence
  Transducible material
MGSSHHHHHHSSGLVPRGSHMAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFLESGGGGSPGRRRRRRRRRRR